(19) United States
(12) Patent Application Publication
(10) Pub. No.: US 2006/0259100 A1
(43) Pub. Date: Nov. 16, 2006

(54) Light therapy device

(76) Inventor: Andreas Hilburg, Oberhausen (DE)

Correspondence Address: MUIRHEAD AND SATURNELLI, LLC, 200 FRIBERG PARKWAY, SUITE 1001, WESTBOROUGH, MA, 01581, US

(21) Appl. No.: 10/542,470

(22) PCT Filed: Jan. 14, 2004
(86) PCT No.: PCT/EP04/00181

§ 371 Date: May 10, 2006

(30) Foreign Application Priority Data
Jan. 17, 2003 (DE) 103 01 867.0

(51) Int. Cl. A61N 5/06

(52) U.S. Cl. 607/88

(57) ABSTRACT

A device for generating luminous stimuli for the eye, comprising a housing that can be fixed in front of the user's face, at least one light source that is disposed inside the housing, and a cover for the light source, which faces the user's eye. The aim of the invention is to further develop said device in such a way that the ability to vary the light is optimized when light is individually directed to a user's eye or eyes. Said aim is achieved by the fact that the cover for the light source is embodied as a luminescent screen made of a diffuse translucent material while three differently colored light sources the illumination of which can be separately regulated, are arranged behind the luminescent screen.

The invention relates to a device for generating luminous stimuli for the eye, comprising a housing, which can be fixed in front of the user's face, at least one light source disposed in the housing and a cover for the light source facing the user's eye.

Goggle-like devices with light sources arranged directly in front of the eyes are known, for example, from the German utility model DE 67 50 962 U and the American patents U.S. Pat. Nos. 4,315,502 and 6,350,275. The utility model DE 67 50 962 U discloses a housing which is similar to swimming goggles. A beaker-type colour filter with a light-source behind it is arranged in front of each eye. The two parts of the housing are adapted to lie on the closed eyelids. Therefore, this device is used to generate pulsed luminous stimuli of one colour through closed eyelids. U.S. Pat. No. 4,315,502 describes a goggle-like device with luminous rings in front of each eye. Several light sources are arranged in each luminous ring. Light pulses are generated of an intensity which can be varied in synchronism or individually for each eye. The U.S. Pat. No. 6,350,275 discloses a goggle-like device in which LEDs (light-emitting diodes) deliver light directly to a subject's retina whereby the LEDs preferably emit green to blue light. This device is also restricted to the pulsation or variation of the intensity of the light.

The object of the present invention is to develop further the device described at the start in such a way that the ability to vary the light is optimised when light is individually directed to a user's eye or eyes.

This object is achieved according to the invention in that the cover for the light source comprises a luminescent screen made of a diffuse translucent material and that three differently coloured light sources, the illumination of which can be separately regulated, are arranged behind the luminescent screen.

The subject of the invention is a device that can be fixed directly in front of the user's eyes similarly to spectacles or snow goggles. Therefore, for the purposes of this application the term “housing” includes both spectacle frames and hood-like structures similar to snow goggles or a pair of separate hood-like eye caps similar to swimming goggles. The housing can be used to a large extent independently of the location and fixed and worn on head whatever the user's attitude and position. The light can be generated with very low energy, since the diffuse translucent screen is located close in front of the user's eyes. Unlike known goggle-type devices for light therapy, this device permits not only the variation of the illumination of the light falling on the eye but also a change of the colour of the light. The colour generated on the diffuse translucent luminescent screen is determined by the respective intensities of the three differently coloured light sources. The colour is created in accordance with the principle of additive colour mixing.

A device permitting the generation of different hues on a luminescent screen according to the principle of additive colour mixing is known in principle, for example from DE 198 09 871 C2. This describes a colour-mixing attachment for the additive colour mixing of the light from three light sources which in the interior of housing has two or more sequential intermediate walls made of a white, diffuse-translucent material and an intermediate hollow section. This colour-mixing attachment is generally intended for illumination technology, for example in discotheques and on concert and theatrical stages, etc., and is intended to permit the generation of spatially homogeneous monochrome light in accordance with the principles of additive colour mixing. Due to its complex construction, it can only be designed as an extremely heavy and bulky unit.

In one practical embodiment of the invention, the housing is opaque and open on one side with the open side being designed to lie against a section of the user's head surrounding at least one eye. In this way, the housing covers at least one of the user's eyes by substantially lying flush against the area of the head surrounding the eye. In practice, the housing preferably lies on the user's face in the style of snow goggles or ski goggles. The housing is opaque so that the eye surrounded by the housing is screened against extraneous light. Inside the housing, the luminescent screen is arranged in front of the user's eye. In this way, placing the housing in front of one of the user's eyes excludes all extraneous light with the luminescent screen on which the coloured light is generated being placed directly in front of the eye.

In a simple embodiment, the light-generating device can be equipped with a single light source which irradiates the luminescent screen. The light source can generate white or coloured light. A device of this kind permits the generation of pulsating monochrome light desired for specific types of therapy. In one versatile embodiment, each luminescent screen is irradiated by three light sources whose colours are mixed additively. In this way, different colours and different intensities of light can be generated on the luminescent screen.

In one practical embodiment, the open side of the housing encloses both of the user's eyes and a luminescent screen is arranged in front of each of the user's eyes. A device of this kind is optimally suited for light therapy and colour therapy, i.e. for treating a patient by the application of light of a specific intensity and specific colour. The intensity and colour of the light can be varied in a prespecified rhythm. Light therapies and colour therapies of this kind can be combined very effectively with magnetotherapies, electrostimulation therapies or audio therapies and are used generally for relaxation and to promote the patient's welfare. In the past, either specific therapy rooms or therapy devices were used for the performance of light therapies. This meant that users were unable to leave the installation site of the therapeutic device and sometimes had to adjust their posture to the device. The combination of light therapy with other therapies was in principle only possible if a combination therapeutic device was installed.

The invention enables the provision of an extremely light, portable housing which may be fixed directly to the user's head in a way similar to snow goggles. This device can be used in conjunction with any other therapeutic devices and in any physical posture of the user.

In one practical embodiment, the housing substantially comprises a front wall and a circumferential ridge which may be placed against the user's forehead, cheeks and bridge of the nose and extends in the edge area of the front wall. The free edge of the ridge encloses the open side of the housing and in one practical embodiment can be provided with an elastically deformable, substantially opaque seal. A seal of this kind prevents hard sections of the housing coming into contact with the user's face. At the same time, it ensures that the ridge lying against the face is adapted to the exact shape of the face. This prevents any the penetration of extraneous light between the face and the ridge since the seal itself is substantially opaque. A seal of this kind is also known with ski goggles or snow goggles.

In order to fix the housing to a user's head, in one practical embodiment a rubber band is provided as a fixing device, which extends with a certain elastic tension behind the user's head of the user. If the housing is embodied similarly to a sunglasses' frame, the fastening device for the housing can be formed by the temples of the glasses.

In the front wall of the housing, preferably an opening is arranged in front of each eye which, with the housing lying on the face as intended, lies in front of the user's eyes and is filled with the luminescent screen.

In one practical embodiment, a cap made of diffuse translucent material, preferably a milky white plastic film, forms the luminescent screen. The cap has a front surface and a sidewall surrounding the periphery of the front surface. An annular collar is attached to the sidewall. The front surface forms said luminescent screen. The light sources are connected to the annular collar so that the extension of the sidewall between the annular collar and the front surface defines the distance of the light sources from the luminescent screen.

With this arrangement, the light sources are on the side of the front wall facing away from the user's face behind the luminescent screen formed by the cap.

With the device according to the invention, the light sources can, as known from prior art, be formed from light-emitting diodes. Arranged behind every luminescent screen are at least one red, one green and one blue light-emitting diode in one practical embodiment. Preferably, diametrically opposed diode pairs are distributed at regular angular distances in a circle around the middle of the luminescent screen.

In practice, a connecting cable with electrical conductors for controlling the light sources can be fixed to the housing. Fastened to the free end of the connecting cable is a connector plug which may be connected to a control unit. The housing can naturally also be connected to the control unit by cableless means, for example by infrared data transmission or a radio link, for example in accordance with the Bluetooth standard. In this case, it makes sense for an energy source to be accommodated in the housing, for example a battery or a rechargeable accumulator to supply the light sources and the data transmission means with power.

The control unit generates current pulses which excite the light sources to light up with a specific intensity according to a prespecified program sequence. In this way, the control unit generates on the luminescent screens the light pulses with a prespecified colour, intensity and duration required to achieve the therapeutic effect. If the device according to the invention is used in conjunction with other therapeutic devices (for example, magnetotherapy, audio therapy, etc.), the control unit can also control the other therapeutic devices and forward electrical signals for the generation of noise or magnetic fields to the relevant devices.

As already mentioned, the device according to the invention comprises an opaque housing with openings in the front wall through which the cap-like luminescent screens protrude. The electronic components are arranged on the rear of the caps and the front wall facing away from the face. In order to ensure that during the light therapy exclusively light emitted by light-emitting diodes reaches the rear of the luminescent screens, one practical embodiment of the device has an opaque covering layer covering the front wall, the light sources and all the other components of the device.

A practical embodiment of the invention will be described hereafter with reference to the attached drawings in which:

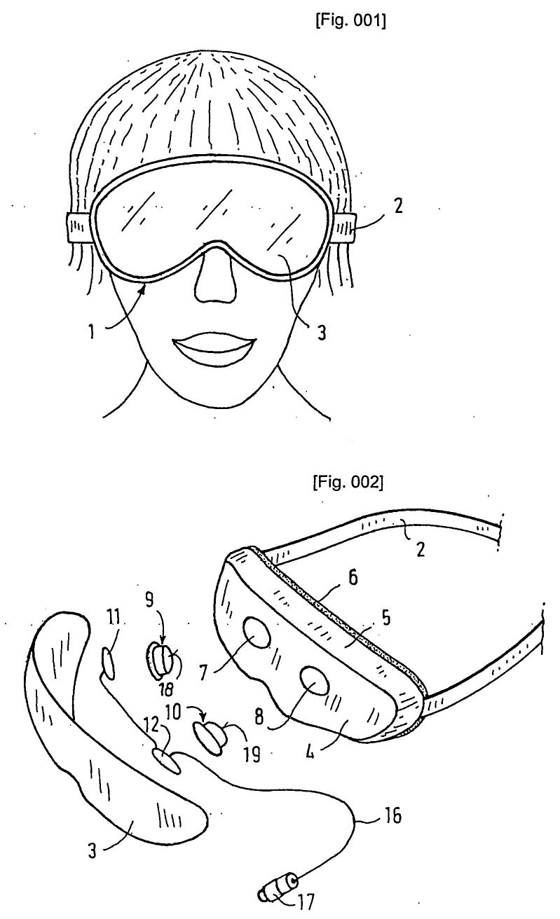

FIG. 1 shows a front view of a device according to the invention attached to a user's head,

FIG. 2 shows components of the device in FIG. 1 in a diagrammatic representation,

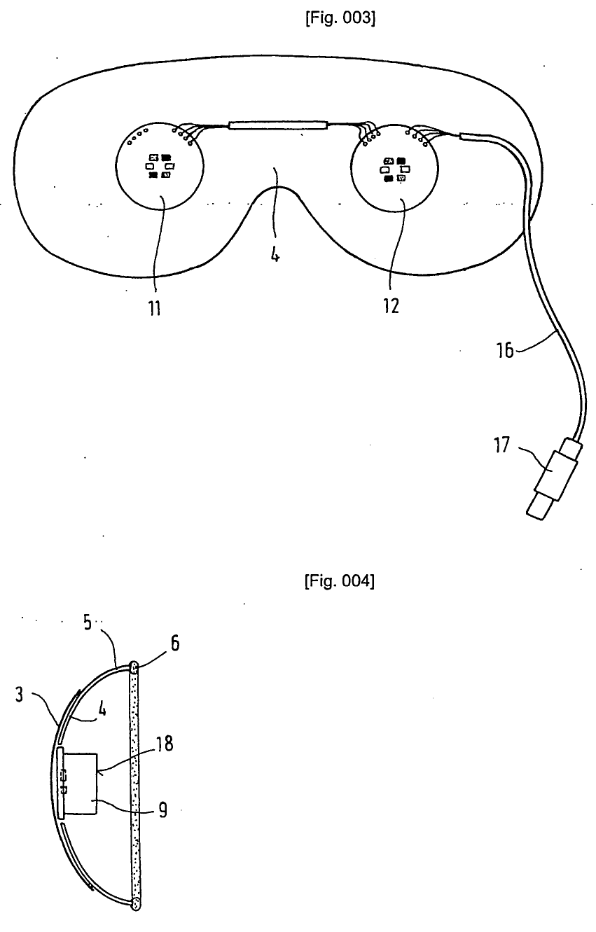

FIG. 3 shows a front view of the device shown in FIG. 1 with the covering layer removed,

FIG. 4 shows a sectional side view of the device in the preceding figures,

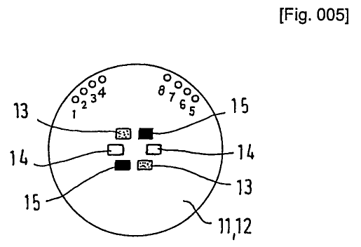

FIG. 5 shows a schematic representation of the light sources assigned to each luminescent screen.

As FIG. 1 shows, the device according to the invention is substantially in the shape of snow goggles or ski goggles. It substantially comprises a housing 1 which is open on one side, which lies on the cheeks, the bridge of the nose and the forehead of the user's face. Arranged on the housing 1 is a rubber band 2 as a fastening device. The rubber band 2 is tensioned with a specific prestress around the user's head and holds the device according to the invention in position. The external surface of the housing 1 visible from the front is formed by an opaque covering layer 3. The covering layer 3 preferably comprises a black, opaque plastic film.

FIG. 2 shows that the housing 1 itself substantially comprises a front wall 4 and a circumferential ridge 5 moulded on the edge of the front wall 4. FIG. 4 shows that the front wall 4 can pass continuously through a material curvature into the circumferential ridge 5.

The front wall 4 and the ridge 5 preferably consist of black, opaque plastic.

Attached to the edge of the circumferential ridge 5 lying on the user's face is a microcellular rubber seal 6 which permits a tight fit on the user's face.

Two openings 7,8 are provided in the front wall 4 of the housing. Two caps 9,10 consisting of milky white diffuse translucent plastic penetrate the openings 7,8.

On each side of the caps 9,10 facing away from the face there is a circular electronic circuit 11,12 on which the light sources are arranged. The light sources are easiest to identify in FIG. 5. Six light-emitting diodes 13,14, 15 are arranged in the central area of the electronic circuits 11,12, which is substantially opposite to the central area of the caps 9,10. Two diametrically opposed light-emitting diodes 13 emit light of a blue colour. Two diametrically opposed light-emitting diodes 14 emit light of a green colour. Two diametrically opposed light-emitting diodes 15 emit light of a red colour.

The intensity of the light emitted by each light-emitting diode 13, 14, 15 substantially corresponds to the intensity of the current flowing through the respective light-emitting diode 13, 14, 15. The current for controlling the light-emitting diodes 13, 14, 15 is specified by a control unit which is connected to the device according to the invention via a connecting cable 16 by means of a connector plug 17.

The light emitted by the light-emitting diodes 13, 14, 15 falls on the front surface 18, 19 of the cap and generates here by means of additive colour mixing a light with a colour and brightness determined by the brightness of the light emitted by the light-emitting diodes 13, 14, 15. The distance between the light-emitting diodes 13, 14, 15 and the front surface 18, 19 of the cap forming the luminescent screen should be selected so that substantially the entire front surface of the cap is irradiated by each light-emitting diode 13, 14 and 15 in order to achieve an as homogeneous as possible colour mixing.

### LIST OF REFERENCE NUMBERS

1 Housing
2 Fastening device, rubber band
3 Covering layer
4 Front wall
5 Ridge
6 Seal
7 Opening
8 Opening
9 Cap
10 Cap
11 Electronic circuit
12 Electronic circuit
13 Blue light-emitting diode, light source
14 Green light-emitting diode, light source
15 Red light-emitting diode, light source
16 Connecting cable
17 Connector plug
18 Luminescent screen, front surface
19 Luminescent screen, front surface

1. A device for generating luminous stimuli for the eye, comprising.
a housing which can be fixed in front of the user's face, at least one light source disposed in the housing and a cover for the light source facing the user's eye, wherein the cover for the light source is a luminescent screen made of a diffuse translucent material and three differently colored light sources, the illumination of which can be separately regulated, are arranged behind the luminescent screen.

2. The device according to claim 1, wherein the housing is provided with a fastening device for fixing in front of the face of the user and that the luminescent screen is arranged inside the housing fixed in this way in front of the user's eye.

3. The device according to claim 1, wherein the housing is opaque and open on one side, with its open side being designed to lie on a section of the user's head surrounding at least one eye.

4. The device according to claim 1, wherein the open side of the housing is designed to lie on a section of the user's face surrounding both eyes and that a luminescent screen is arranged in front of each of the user's eyes.

5. The device according to claim 1, wherein the housing comprises a front wall and a ridge which can lie on the user's forehead, cheeks and bridge of the nose of the user which extends in the edge area of the front wall.

6. The device according to claim 5, wherein a free edge of the ridge enclosing the open side of the housing is provided with an elastically deformable, substantially opaque seal.

7. The device according to claim 1, wherein a rubber band for fixing the housing to the user's head is arranged on the housing.

8. The device according to claim 1, wherein an opening is arranged in the a front wall of the housing which, with the housing fixed to the head as intended, lies in front of the user's eye and which is filled by the luminescent screen.

9. The device according to claim 8, further comprising at least one cap made of diffuse translucent material which comprises a front surface, a sidewall surrounding the periphery of the front surface and an annular collar, whereby the front surface forms the luminescent screen.

10. The device according to claim 1, wherein the light sources lie on the side of the front wall facing away from the user's face behind the luminescent screen.

11. The device according to claim 1, wherein the light sources are formed by light-emitting diodes.

12. The device according to claim 11, wherein at least one red, one green and one blue light-emitting diode are arranged behind every luminescent screen.

13. The device according to claim 1, wherein a connecting cable is attached to the housing with electrical conductors for controlling the light sources.

14. The device according to claim 13, wherein a connecting plug for connection to a control unit for the light sources is arranged at the free end of the connecting cable.

15. The device according to claim 1, wherein an opaque covering layer covers the front wall and the light sources.

16. A device for generating luminous stimuli, comprising:
a housing;
at least three differently colored light sources disposed in the housing;
at least one cover for the light sources, wherein the at least one cover for the light sources is a luminescent screen made of a diffuse translucent material, wherein said at least three differently colored light sources are disposed behind the luminescent screen, and wherein illumination of each of said at least three differently colored light sources are separately controlled.

17. The device according to claim 16, further comprising:
a control unit electrically connected to said at least three differently colored light sources and that controls said at least three differently colored light sources.

18. The device according to claim 16, further comprising:
an opaque covering layer that covers a front wall of said housing.

19. The device according to claim 16, further comprising:
a first area disposed in said housing that includes a first set of said at least three differently colored light sources disposed behind a first luminescent screen; and
a second area disposed in said housing that includes a second set of said at least three differently colored light sources disposed behind a second luminescent screen.

20. The device according to claim 16, wherein said housing is a set of goggles.

21. A method for generating light stimuli, comprising:
disposing at least three differently colored lights in a housing;
disposing at least one cover over said at least three differently colored lights, wherein said at least one cover is a luminescent screen made of a diffuse translucent material; and
separately controlling illumination of said at least three differently colored lights.
